(19) United States
(12) Patent Application Publication
(10) Pub. No.: US 2017/0291870 A1
(43) Pub. Date: Oct. 12, 2017

(54) SURFACTANT-CONTAINING AMIDE COMPOUND SOLUTION

(71) Applicant: Mitsubishi Chemical Corporation, Tokyo (JP)

(72) Inventors: Makoto KANO, Tokyo (JP); Norifumi HAGIYA, Tokyo (JP)

(73) Assignee: Mitsubishi Rayon Co., Ltd., chiyoda-ku (JP)

(21) Appl. No.: 15/508,350

(22) PCT Filed: Feb. 15, 2016
(86) PCT No.: PCT/JP2016/054245

§ 371 Date: Mar. 2, 2017

(30) Foreign Application Priority Data
Mar. 2, 2015 (JP) 2015-039814

(51) Int. Cl. C07C 233/09; C12P 13/02; C08F 120/56

(52) U.S. Cl. CPC C07C 233/09; C08F 120/56; C12P 13/02

(57) ABSTRACT

The present invention relates to an amide compound solution comprising an amide compound and a surfactant. More specifically, the invention relates to an amide compound solution comprising amide compound, and 2.7˜20 mg of a cationic surfactant per 1 kg of the amide compound or 0.01˜10 mg of a C15˜C20 carboxylic acid or its salt as an anionic surfactant per 1 kg of the amide compound. The present invention provides an amide compound solution which is manufactured using a biocatalyst and which has a low level of foaming, and thereby improving the operability and yield when manufacturing an amide compound-based polymer.

### TECHNICAL FIELD

The present application is based upon and claims the benefit of priority to Japanese Patent Application No. 2015-039814, filed Mar. 2, 2015, the entire contents of which are incorporated herein by reference.

The present invention relates to a solution of an amide compound such as acrylamide, more specifically, to an amide compound solution containing a surfactant.

### BACKGROUND ART

Polymers of amide compounds such as acrylamide are used in a wide variety of applications, for example, flocculants and oil recovery agents, as well as strength enhancers and thickeners in the paper industry. Amide compounds are important substances as the material for forming such polymers.

In the past, to industrially manufacture acrylamide, a sulfuric acid hydrolysis process was employed for preparing an acrylamide sulfate solution by mixing acrylonitrile with sulfuric acid and water, followed by heating the mixture. Then, methods for industrially manufacturing acrylamide were shifted to using a copper catalyst for preparing an acrylamide solution by hydrating acrylonitrile in the presence of a copper catalyst such as metallic copper, reduced copper and Raney copper.

Moreover, biocatalytic methods, as manufacturing methods that generate only a small amount of byproducts, have been moving into the industrial mainstream in recent years. In biocatalytic methods, solutions of amide compounds such as acrylamide are obtained by using biocatalysts, for example, nitrile hydratase derived from microorganisms.

However, since impurities such as proteins and sugars derived from the biocatalyst are also contained in an amide compound solution prepared by a biocatalytic method, the amide compound solution tends to foam. Thus, it is difficult to handle an amide compound solution when the solution is transferred, transported, or stored. Furthermore, when an amide compound-based polymer is formed by polymerizing an amide compound, the amide compound solution foams and overflows from the polymerization vessel, thus lowering the yield of the amide compound-based polymer.

Accordingly, a low level of foaming is desired for amide compound solutions manufactured using biocatalysts. Since proteins derived from biocatalysts cause foaming, to suppress amide compound solutions from foaming, it is proposed to remove proteins by bringing the amide compound solutions into contact with active carbon having a predetermined specific surface area (Patent Literature 1).

### CITATION LIST

### Patent Literature

Patent Literature 1: JP2012-62268A

### SUMMARY OF THE INVENTION

### Problems to be Solved by the Invention

However, in the method described in Patent Literature 1, active carbon needs to be added to and then removed from the amide compound solution. It is also necessary to install a specific apparatus or system for processing the amide compound solution with the active carbon. Moreover, since making contact with amide compound solutions causes clogging in active carbon, the active carbon needs to be reactivated, which in turn requires an installment of a specific apparatus or system for that purpose. Accordingly, the steps of manufacturing an amide compound solution are complicated, and such a method is not preferable for industrial production.

Considering the above, the primary objective of the present invention is to provide an amide compound solution which is manufactured using a biocatalyst and which has a low level of foaming.

### Solutions to the Problems

The inventors of the present invention have carried out intensive studies while considering the problems in conventional technology and found that the above-mentioned objective is achieved when a cationic or anionic surfactant (C15˜C20 carboxylic acid or its salt) with a specific concentration is present in a solution of an amide compound such as acrylamide. Accordingly, the present invention is completed.

Namely, the present invention has the following aspects (1)˜(12):
(1) An amide compound solution, containing an amide compound and 2.7˜20 mg of a cationic surfactant per 1 kg of the amide compound or 0.01˜10 mg of a C15˜C20 carboxylic acid or its salt as an anionic surfactant per 1 kg of the amide compound;
(2) The amide compound solution according to (1) above, containing 2.7˜20 mg of a cationic surfactant per 1 kg of the amide compound;
(3) The amide compound solution according to (1) or (2) above, in which the cationic surfactant is at least one type selected from among benzethonium chloride, benzalkonium chloride, cetylpyridinium chloride and dequalinium chloride;
(4) The amide compound solution according to any of (1)˜(3) above, in which the cationic surfactant is at least one type selected from benzethonium chloride and benzalkonium chloride;
(5) An amide compound solution, containing 15˜150 mg of a cationic surfactant per 1 gram of protein in the amide compound solution;
(6) The amide compound solution according to (1) above, containing 0.01˜10 mg of a C15˜C20 carboxylic acid or its salt as an anionic surfactant per 1 kg of the amide compound;
(7) The amide compound solution according to (6) above, in which the anionic surfactant is at least one type selected from among pentadecylic acid, palmitic acid, margaric acid, stearic acid, arachidic acid and their salts;
(8) An amide compound solution, containing 0.02˜100 mg of an anionic surfactant per 1 gram of protein in the amide compound solution;
(9) The amide compound solution according to any of (1)˜(8) above, in which the amide compound is produced by hydrating a nitrile compound with a biocatalyst;
(10) The amide compound solution according to any of (1)˜(9) above, in which the concentration of the amide compound in the amide compound solution is set at 25˜60 mass %;
(11) The amide compound solution according to any of (1)˜(10) above, in which the amide compound is acrylamide; and
(12) A method for manufacturing an amide compound-based polymer by polymerizing the amide compound in an amide compound solution described in any of (1)˜(11) above.

### Effects of the Invention

According to the present invention, when a cationic or anionic surfactant (C15˜C20 carboxylic acid or its salt) with a specific concentration is present in an acrylamide solution, the level of foaming is reduced in the acrylamide solution. Accordingly, handling is easier when the acrylamide solution is transferred, transported, stored or used in a polymerization process.

### DETAILED DESCRIPTION OF THE EMBODIMENTS

In the following, the present invention is described in detail.

### (1) Biocatalyst

The amide compound solution related to the present invention is preferred to be manufactured by a biocatalytic method, since a highly pure amide compound is obtained with a lower amount of reaction byproducts. Manufacturing an amide compound by using a biocatalyst is not limited to any specific method as long as an amide compound is produced from the corresponding nitrile compound by the use of hydratase such as nitrile hydratase. The type of enzyme or microorganism, reaction conditions or the like may be appropriately selected by a person skilled in the art. For example, the method described in WO2009/113654 may be employed.

A biocatalyst for producing the amide compound solution may be animal cells, plant cells, organelles, bacterial cells (living or dead) or their treated products, containing enzymes that work as catalysts for desired reactions.

Examples of treated enzyme products are crude enzymes extracted from cells or purified enzymes thereof, along with animal cells, plant cells, organelles, bacterial cells (living or dead) or enzymes themselves that are immobilized by entrapment methods, crosslinking methods, carrier binding methods or the like.

Entrapment methods are for entrapping bacterial cells or enzymes into fine mesh of polymer gels, or coating them with a semitransparent polymer membrane. Crosslinking methods are for crosslinking enzymes with reagents having two or more functional groups (multifunctional crosslinking agent). In addition, carrier binding methods are for binding enzymes to water-insoluble carriers.

Immobilization carriers used for immobilizing enzymes or cells are glass beads, silica gel, polyurethane, polyacryl amide, polyvinyl alcohol, carrageenan, alginic acid, agar, gelatin or the like.

Examples of bacterial cells are microorganisms belonging to genus Nocardia, genus Corynebacterium, genus Bacillus, genus Pseudomonas, genus Micrococcus, genus Rhodococcus, genus Acinetobacter, genus Xanthobacter, genus Streptomyces, genus Rhizobium, genus Klebsiella, genus Enterobacter, genus Erwinia, genus Aeromonas, genus Citrobacter, genus Achromobacter, genus Agrobacterium, genus Pseudonocardia, and the like.

As for the enzyme, nitrile hydratases produced by the microorganisms listed above, for example, may be used.

### (2) Manufacturing Amide Compound

An amide compound may be manufactured by using a biocatalyst through continuous reaction (for continuously producing an amide compound) or batch reaction (for non-continuously producing an amide compound). From the viewpoint of production efficiency, a continuous reaction method is preferred.

Here, a method for continuous reaction means an amide compound is continuously produced by carrying out a continuous or intermittent supply of reaction materials (including water, biocatalyst and nitrile compound) and a continuous or intermittent retrieval of the reaction mixture (including the produced amide compound), without completely retrieving all the reaction mixture in the reaction vessel.

The amount of a biocatalyst is not limited specifically as long as it is capable of efficiently producing an amide compound, and a person skilled in the art may select the amount appropriately based on the type and state of the biocatalyst. For example, the activity of a biocatalyst to be supplied into a reaction vessel is preferred to be adjusted at approximately 50˜500 U per 1 mg of the dried bacterial cells at a reaction temperature of 10° C. The unit “U” means the activity of producing an amide compound from the corresponding nitrile compound at a rate of 1 μmol/min.

During the reaction, the nitrile compound concentration in the reaction mixture may vary depending on the type and state of the biocatalyst to be used, but it is preferred to be approximately 0.5˜15 mass %.

The concentration of an amide compound solution to be produced is not limited specifically, and may be selected appropriately according to usage purposes or the like. For example, the concentration of an amide compound solution is preferred to be 25˜60 mass %, more preferably 30˜55 mass %.

By setting the amide compound concentration in an amide compound solution to be at least 25 mass %, the storage tank volume is reduced and transportation costs are thereby suppressed. By setting the amide compound concentration to be no greater than 60 mass %, crystallization of the amide compound at approximately room temperature is prevented. Accordingly, an increase in equipment cost caused by an additional heating device is prevented. Also, a complication of operational procedures caused by additional temperature control is prevented.

After the reaction is completed, the biocatalyst in the amide compound solution is removed, if applicable. To remove the biocatalyst from an amide compound solution, for example, filtration, centrifugation, flocculation, adsorption or the like may be used.

The “amide compound” related to the present invention is not limited to any specific compound, but amide compounds having unsaturated bonds for forming polymers are highly preferable in industrial applications. Such amide compounds having unsaturated bonds are, for example, monoamide compounds such as acrylamide, methacrylamide, nicotinamide, crotonamide, tiglic amide, 2-pentenoic acid amide, 3-pentenoic acid amide, 4-pentenoic acid amide, 2-hexenoic acid amide, 3-hexenoic acid amide, and 5-hexenoic acid amide, diamide compounds such as fumaric acid diamide, maleic acid diamide, citraconic acid diamide, mesaconic acid diamide, itaconic acid diamide, 2-pentenoic diacid diamide, and 3-hexenoic diacid diamide; and so on. Among them, it is preferred to use monoamide compounds, more preferably, acrylamide and methacrylamide. In the present application, acrylamide and methacrylamide may collectively be referred to as “(meth)acrylamide.”

### (3) Cationic Surfactant

The amide compound solution related to the present invention may also contain a cationic surfactant. The content of a cationic surfactant is preferred to be at least 2.7 mg, more preferably at least 3.0 mg, even more preferably at least 3.5 mg, per 1 kg of the amide compound.

When multiple types of cationic surfactants are contained in an amide compound solution, the total amount of the multiple cationic surfactants is set to be at least 2.7 mg, more preferably at least 3.0 mg, even more preferably at least 3.5 mg, per 1 kg of the amide compound.

By setting the content of cationic surfactant in an amide compound solution to be at least 2.7 mg per 1 kg of the amide compound, foaming of the amide compound solution is sufficiently suppressed.

The upper limit of the cationic surfactant content in an amide compound solution is not limited specifically; however, from the viewpoint of quality and cost performance, it is preferred to be no greater than 20 mg, more preferably no greater than 15 mg, even more preferably no greater than 10 mg, per 1 kg of the amide compound.

To introduce a cationic surfactant in an amide compound solution is not limited to any specific method, and the cationic surfactant may simply be added to the amide compound solution. At that time, a cationic surfactant may be added as is, or may be added after it is made into a cationic surfactant solution.

In addition, a cationic surfactant may be added in any of the steps for producing an amide compound, for example, a step for preparing a biocatalyst, a step for producing an amide compound by hydrating a nitrile compound in the presence of a biocatalyst, a step for purifying the amide compound solution, or a step for storing the amide compound solution. The cationic surfactant may be added in two or more steps above.

Moreover, when a cationic surfactant is already present in an amide compound solution in a production step but the content is less than 2.7 mg per 1 kg of the amide compound, the cationic surfactant may be added so as to make its content 2.7 mg or greater.

Confirming the amount of the cationic surfactant in an amide compound solution is not limited to any specific method, and liquid chromatography-mass spectrometry or the like may be employed.

The cationic surfactant used in the embodiments of the present invention is not limited to any specific type as long as it has a cationic hydrophilic group. Examples are benzethonium chloride, benzalkonium chloride, cetylpyridinium chloride, dequalinium chloride, and the like. Among them, benzethonium chloride and benzalkonium chloride are preferred. They may be used alone or in combination thereof.

In addition, the amount of cationic surfactant in the embodiments of the present invention may be set based on the amount of protein in the amide compound solution. For example, the amount of a cationic surfactant to be added (to be contained) is preferred to be 15˜150 mg, more preferably 16˜145 mg, even more preferably 18˜140 mg, per 1 gram of protein in the amide compound solution.

In the present application, measuring the amount of protein in an amide compound solution is not limited to any specific method, and any known method, for example, the Lowry method, may be used.

### (4) Anionic Surfactant

The surfactant in the amide compound solution related to the present invention may be an anionic surfactant; for example, C15˜C20 carboxylic acids or their salts may be used.

C15˜C20 carboxylic acids may be saturated or unsaturated aliphatic acids, but saturated aliphatic acids are preferred. Among the carboxylic acids, at least one type selected from among pentadecylic acid, palmitic acid, margaric acid, stearic acid and arachidic acid is preferred, more preferably stearic acid.

In addition, the elements for forming salts with C15˜C20 carboxylic acids are alkali metals such as sodium and potassium and alkaline earth metals such as magnesium and calcium.

The content of an anionic surfactant is preferred to be 0.01˜10 mg, more preferably 0.02˜9 mg, even more preferably 0.03˜8 mg, most preferably 0.05˜1 mg per 1 kg of the amide compound. When the content of an anionic surfactant is at least 0.01 mg per 1 kg of the amide compound, a sufficient defoaming effect is achieved. In addition, the lower limit of an anionic surfactant is set to be no greater than 10 mg per 1 kg of the amide compound, because the amount beyond that does not contribute to obtaining any further significant effects.

In addition, the amount of anionic surfactant in the embodiments of the present invention may be set based on the amount of protein in the amide compound solution. For example, the amount of anionic surfactant to be added (to be contained) is preferred to be 0.02˜100 mg, more preferably 0.04˜95 mg, even more preferably 0.06˜90 mg, per 1 gram of protein in the amide compound solution.

In the present application, measuring the amount of protein in an amide compound solution is not limited specifically, and any known method, for example, the Lowry method, may be used.

Since the amide compound solution related to the present invention has a lower level of foaming, handling is easier when the solution is transferred, transported, stored or used for manufacturing amide compound polymers. Moreover, since overflow from the polymerization vessel caused by foaming of the amide compound solution is suppressed, a decrease in the yield is prevented when an amide compound-based polymer is produced from the amide compound.

Furthermore, a cationic or anionic surfactant will hardly affect the quality of the amide compound or amide compound-based polymer manufactured by using the surfactant. Namely, the amide compound solution with a lower level of foaming related to the present invention has the same degree of quality as that of an amide compound solution produced without using a cationic surfactant. Also, the amide compound-based polymers produced using their respective amide compounds show the same degree of quality.

### (5) Method for Producing Amide Compound-Based Polymer

The present invention also provides a method for producing amide compound-based polymers such as poly(meth)acrylamide by using an amide compound solution containing a surfactant. The method may be homopolymerizing the amide compound, or copolymerizing the amide compound with one or more other monomers.

Examples of copolymerizable monomers are unsaturated carboxylic acids such as acrylic acid, methacrylic acid, maleic acid, fumaric acid, itaconic acid and their salts; vinylsulfonic acid, styrenesulfonic acid and acrylamidomethylpropanesulfonic acid, or their salts; alkylaminoalkyl esters of (meth)acrylic acid or their derivatives; N,N-dialkylaminoalkyl (meth)acrylamide or its derivatives; hydrophilic acrylamides such as acetone acrylamide and N-propyl acrylamide; (meth)acrylate derivatives such as methyl (meth)acrylate and ethyl (meth)acrylate; and olefins such as acrylonitrile, methacrylonitrile, vinyl acetate, vinyl chloride, vinylidene chloride, ethylene, propylene and butene.

Polymerization is carried out by adding a polymerization initiator to a solution containing the monomers as raw materials (solution containing an amide compound and other monomers) under appropriate conditions. It may be any normally employed method, for example, solution polymerization, suspension polymerization or emulsion polymerization.

A radical polymerization initiator may be used. Examples of a radical polymerization initiator are peroxides such as potassium persulfate, ammonium persulfate, hydrogen peroxide and benzoyl peroxide; free-radical azo initiators such as azobisisobutyronitrile and azobis-(2-amidinopropane)dichloride; so-called redox catalysts formed in combination with the above peroxide and a reducing agent such as sodium bisulfite, triethanolamine and ferrous ammonium sulfate. Those polymerization initiators may be used alone or in combination thereof.

According to the present invention, when an amide compound is produced by using a biocatalyst, an amide compound solution with a low level of foaming is obtained, and handling is thereby easier when the amide compound solution is transferred, transported, stored or used for a polymerization process to produce an amide compound-based polymer. Furthermore, since overflow of the amide compound solution from the reaction vessel is suppressed, a decrease in the yield is prevented when an amide compound-based polymer is produced using the amide compound.

### EXAMPLES

In the following, the present invention is described in further detail. However, the present invention is not limited to those examples.

### Example 1

### (1) Measuring Concentration of Cationic Surfactant in Acrylamide Solution

The concentration of benzethonium chloride in a commercially available 50 mass % acrylamide solution (made by Mitsubishi Rayon Co., Ltd., manufactured by hydrating acrylonitrile using a biocatalyst, pH 6.8) was determined by liquid chromatography.

For analysis, HPLC Alliance 2695 (made by Waters) as the HPLC system and ZORBAX Eclipse XDB-C18 (5 μm, 4.6×150 mm, made by Agilent Technologies) as the column were used. The mobile phase was 100 mM NaCl/methanol=20/80 with a flow rate of 1.0 m/min, into which 10 μL of a sample was injected. A PDA 2996 detector (made by Waters) was used for detection.

Accordingly, it was found that 2.3 grams of benzethonium chloride per 1 kg of acrylamide was contained.

### (2) Adjusting Content of Cationic Surfactant

The concentration of a cationic surfactant was adjusted to 1000 mg/kg by diluting benzethonium chloride (made by Kanto Chemical Co., Inc., Cica 1st grade) with pure water.

Into 1 kg of the 50 mass % acrylamide solution (namely, 500 grams of acrylamide), 0.25 grams of the 1000 mg/kg benzethonium chloride solution was added and mixed well. Accordingly, an acrylamide solution was prepared, containing 2.8 mg of benzethonium chloride per 1 kg of acrylamide (acrylamide solution 1).

### (3) Foaming Test of Acrylamide Solution

Into a 1000 mL-capacity 25 mm-external diameter cylindrical glass container equipped at the container bottom with an air sparger having a 30 μm hole diameter, 500 mL of acrylamide solution 1 was supplied.

The acrylamide solution was foamed by blowing air from the air sparger for 30 seconds at a rate of 100 mL/min, and the air supply was turned off. The time was measured from the moment the air supply was turned off to the moment the foam of the acrylamide solution disappeared. The result was 5 seconds (a preferred time for the foam to disappear is 10 seconds or less).

### Comparative Example 1

The test was conducted the same as in Example 1 except that benzethonium chloride was not added to the acrylamide solution. As a result, it took 29 seconds for the foam to disappear.

### Example 2

Into 1 kg of the 50 mass % acrylamide solution used in Example 1, 0.85 grams of the 1000 mg/kg benzethonium chloride solution prepared in Example 1 was added and mixed well. Accordingly, an acrylamide solution was prepared, containing 4.0 mg of benzethonium chloride per 1 kg of acrylamide (acrylamide solution 2).

Except that acrylamide solution 2 was used, the same process as in Example 1 was conducted for measuring the time it took for the foam to disappear from the acrylamide solution. The result was 4 seconds.

### Comparative Example 2

Into 1 kg of the 50 mass % acrylamide solution used in Example 1, 0.15 grams of the 1000 mg/kg benzethonium chloride solution prepared in Example 1 was added and mixed well. Accordingly, an acrylamide solution was prepared, containing 2.6 mg of benzethonium chloride per 1 kg of acrylamide (acrylamide solution 3).

Except that acrylamide solution 3 was used, the same process as in Example 1 was conducted for measuring the time it took for the foam to disappear from the acrylamide solution. The result was 18 seconds.

### Example 3

Into 1 kg of the 50 mass % acrylamide solution used in Example 1, 1.85 grams of the 1000 mg/kg benzethonium chloride solution prepared in Example 1 was added and mixed well. Accordingly, an acrylamide solution was prepared, containing 6.0 mg of benzethonium chloride per 1 kg of acrylamide (acrylamide solution 4).

Except that acrylamide solution 4 was used, the same process as in Example 1 was conducted for measuring the time it took for the foam to disappear from the acrylamide solution. The result was 4 seconds.

The results of Example 1˜3 and Comparative Examples 1, 2 are all shown in Table 1.

TABLE 1
cationic surfactant: benzethonium chloride

| | Cationic surfactant | Time before foam |
|---|---|---|
| | [mg/kg] | disappears [sec] |
| Example 1 | 2.8 | 5 |
| Example 2 | 4.0 | 4 |
| Example 3 | 6.0 | 4 |
| Comp. Example 1 | 2.3 | 29 |
| Comp. Example 2 | 2.6 | 18 |

### Example 4

Benzalkonium chloride (made by Kanto Chemical Co., Inc., Cica 1st grade) was diluted with pure water to have a 1000 mg/kg concentration.

Into 1 kg of the 50 mass % acrylamide solution used in Example 1, 0.25 grams of the 1000 mg/kg benzalkonium chloride solution was added and mixed well. Accordingly, an acrylamide solution was prepared, containing 2.8 mg of cationic surfactants (the total amount of benzethonium chloride and benzalkonium chloride) per 1 kg of acrylamide (acrylamide solution 5).

Except that acrylamide solution 5 was used, the same process as in Example 1 was conducted for measuring the time it took for the foam to disappear from the acrylamide solution. The result was 6 seconds.

### Comparative Example 3

Into 1 kg of the 50 mass % acrylamide solution used in Example 1, 0.15 grams of the 1000 mg/kg benzalkonium chloride solution prepared in Example 4 was added and mixed well. Accordingly, an acrylamide solution was prepared, containing 2.6 mg of cationic surfactants (the total amount of benzethonium chloride and benzalkonium chloride) per 1 kg of acrylamide (acrylamide solution 6).

Except that acrylamide solution 6 was used, the same process as in Example 1 was conducted for measuring the time it took for the foam to disappear from the acrylamide solution. The result was 23 seconds.

### Example 5

Into 1 kg of the 50 mass % acrylamide solution used in Example 1, 0.85 grams of the 1000 mg/kg benzalkonium chloride solution prepared in Example 4 was added and mixed well. Accordingly, an acrylamide solution was prepared, containing 4.0 mg of cationic surfactants (the total amount of benzethonium chloride and benzalkonium chloride) per 1 kg of acrylamide (acrylamide solution 7).

Except that acrylamide solution 7 was used, the same process as in Example 1 was conducted for measuring the time it took for the foam to disappear from the acrylamide solution. The result was 6 seconds.

### Example 6

Into 1 kg of the 50 mass % acrylamide solution used in Example 1, 1.85 grams of the 1000 mg/kg benzalkonium chloride solution prepared in Example 4 was added and mixed well. Accordingly, an acrylamide solution was prepared, containing 6.0 mg of cationic surfactants (the total amount of benzethonium chloride and benzalkonium chloride) per 1 kg of acrylamide (acrylamide solution 8).

Except that acrylamide solution 8 was used, the same process as in Example 1 was conducted for measuring the time it took for the foam to disappear from the acrylamide solution. The result was 5 seconds.

The results of Examples 4˜6 and Comparative Example 3 are all shown in Table 2.

TABLE 2
cationic surfactant: benzethonium chloride, benzalkonium chloride

| | Cationic surfactant | Time before foam |
|---|---|---|
| | [mg/kg] | disappears [sec] |
| Example 4 | 2.8 | 6 |
| Example 5 | 4.0 | 6 |
| Example 6 | 6.0 | 5 |
| Comp. Example 3 | 2.6 | 23 |

### Example 7

The concentration of an anionic surfactant was adjusted to 1000 mg/kg by diluting a sodium stearate solution (made by Tokyo Chemical Industry Co., Ltd.) with pure water.

Into 1 kg of the 50 mass % acrylamide solution used in Example 1, 0.025 grams of the 1000 mg/kg sodium stearate solution was added and mixed well. Accordingly, an acrylamide solution was prepared, containing 0.05 mg (0.05 ppm) of anionic surfactant per 1 kg of acrylamide (acrylamide solution 9).

Except that acrylamide solution 9 was used, the same process as in Example 1 was conducted for measuring the time it took for the foam to disappear from the acrylamide solution. The result was 5 seconds.

### Examples 8˜11

Except that acrylamide solutions were prepared to respectively contain 0.1 mg, 0.2 mg, 0.5 mg and 1.0 mg (0.1 ppm, 0.2 ppm, 0.5 ppm and 1.0 ppm) of sodium stearate per 1 kg of acrylamide (acrylamide solutions 10˜13), the same process as in Example 7 was conducted for measuring the time it took for the foam to disappear from each of the acrylamide solutions. The results were 7, 7, 4 and 3 seconds respectively.

### Comparative Examples 4, 5

Except that acrylamide solutions were prepared to respectively contain 0.2 mg and 0.5 mg (0.2 ppm, 0.5 ppm) of sodium myristate (made by Tokyo Chemical) per 1 kg of acrylamide (acrylamide solutions 14, 15), the same process as in Example 7 was conducted for measuring the time it took for the foam to disappear from each of the acrylamide solutions. The results were 150 seconds and 200 seconds respectively.

### Comparative Examples 6, 7

Except that acrylamide solutions were prepared to contain 0.2 mg and 0.5 mg (0.2 ppm, 0.5 ppm) of sodium laurate (made by Tokyo Chemical) respectively per 1 kg of acrylamide (acrylamide solutions 16, 17), the same process as in Example 7 was conducted for measuring the time it took for the foam to disappear from each of the acrylamide solutions. The results were 185 seconds and 295 seconds respectively.

The results of Examples 7˜11 and Comparative Examples 4˜7 are all shown in Table 3.

TABLE 3

| | Anionic surfactant | | Time before foam |
|---|---|---|---|
| | [mg/kg] | | disappears [sec] |
| Example 7 | sodium | 0.05 | 5 |
| Example 8 | stearate | 0.1 | 7 |
| Example 9 | | 0.2 | 7 |
| Example 10 | | 0.5 | 4 |
| Example 11 | | 1.0 | 3 |
| Comp. Example 4 | sodium | 0.2 | 150 |
| Comp. Example 5 | myristate | 0.5 | 200 |
| Comp. Example 6 | sodium | 0.2 | 185 |
| Comp. Example 7 | laurate | 0.5 | 295 |

### Comparative Example 8

Except that acrylamide solutions were prepared by replacing the anionic surfactant (sodium stearate) with an alcohol-based defoamer—ADEKANOL LG-295S (made by Adeka Corporation)—at their respective concentrations of 0, 0.1, 0.3, 0.5, 1, 10, 100 and 300 ppm (mg/kg) in acrylamide solution 9 of Example 7, the same process as in Example 7 was conducted for measuring the time it took for the foam to disappear from each of the acrylamide solutions. The results were 500˜600 seconds when the concentration of alcohol defoamer was 1 ppm or less, but no measurement was available when the concentration of alcohol defoamer was 10 ppm or greater. The results in Comparative Example 8 are all shown in Table 4.

### Comparative Example 9

Except that acrylamide solutions were prepared by replacing the anionic surfactant (sodium stearate) with a silicone-based defoamer—Shin-Etsu Silicone KS-604 (made by Shin-Etsu Chemical Co., Ltd.)—at their respective concentrations of 0, 0.3, 1 and 100 ppm (mg/kg) in acrylamide solution 9 of Example 7, the same process as in Example 7 was conducted for measuring the time it took for the foam to disappear from each of the acrylamide solutions. The results were 550, 510, 300 and 400 seconds respectively.

The results in Comparative Example 9 are all shown in Table 4.

TABLE 4

| Defoamer | Time before foam disappears [sec] | |
|---|---|---|
| concentration | Comp. Example 8 | Comp. Example 9 |
| [mg/kg] | Adeka NOL LG-295S | Shin-Etsu Silicone KS-604 |
| 0 | 550 | |
| 0.1 | 500~600 | — |
| 0.3 | 500~600 | 510 |
| 0.5 | 500~600 | — |
| 1 | 500~600 | 300 |
| 10 | unable to measure | — |
| 100 | unable to measure | 400 |
| 300 | unable to measure | — |

### Example 12

When the protein concentration of acrylamide solution 1 used in Example 1 was measured by the Lowry method, it was 76 mg per 1 kg of acrylamide solution. When the concentration of the cationic surfactant used in Example 1 was converted per 1 gram of protein, it was 18.4 mg.

When the protein concentration of acrylamide solution 1 used in Example 7 was measured by the Lowry method, it was 76 mg per 1 kg of acrylamide solution. When the concentration of the anionic surfactant used in Example 7 was converted per 1 gram of protein, it was 0.7 mg.

### INDUSTRIAL APPLICABILITY

According to the present invention, the level of foaming is reduced in solutions of amide compounds such as acrylamide, and handling is thereby easier when amide compound solutions are transferred, transported, stored or used for a polymerization process to produce amide compound-based polymers.

Furthermore, according to the present invention, amide compound solutions are suppressed from overflowing from polymerization vessels, thus preventing a lowered yield when amide compound-based polymers are manufactured using amide compounds.

The contents of all the publications, patent literatures and patent applications cited in the present application are incorporated herein by reference.

1. An amide compound solution, comprising:
an amide compound; and
2.7˜20 mg of a cationic surfactant per 1 kg of the amide compound, or
0.01˜10 mg of a C15˜C20 carboxylic acid or its salt as an anionic surfactant per 1 kg of the amide compound.

2. The amide compound solution according to claim 1, comprising 2.7˜20 mg of a cationic surfactant per 1 kg of the amide compound.

3. The amide compound solution according to claim 2, wherein the cationic surfactant is at least one type selected from among benzethonium chloride, benzalkonium chloride, cetylpyridinium chloride and dequalinium chloride.

4. The amide compound solution according to claim 2, wherein the cationic surfactant is at least one type selected from benzethonium chloride and benzalkonium chloride.

5. An amide compound solution, comprising:
an amide compound; and
15˜150 mg of a cationic surfactant per 1 gram of protein in the amide compound solution.

6. The amide compound solution according to claim 1, comprising 0.01˜10 mg of a C15˜C20 carboxylic acid or its salt as an anionic surfactant per 1 kg of the amide compound.

7. The amide compound solution according to claim 6, wherein the anionic surfactant is at least one type selected from among pentadecylic acid, palmitic acid, margaric acid, stearic acid, arachidic acid or their salts.

8. An amide compound solution, comprising:
an amide compound; and
0.02˜100 mg of an anionic surfactant per 1 gram of protein in the amide compound solution.

9. The amide compound solution according to claim 2, wherein the amide compound is produced by hydrating a nitrile compound with a biocatalyst.

10. The amide compound solution according to claim 2, wherein the concentration of the amide compound in the amide compound solution is set at 25˜60 mass %.

11. The amide compound solution according to claim 2, wherein the amide compound is acrylamide.

12. A method for manufacturing an amide compound-based polymer, comprising:
polymerizing the amide compound in an amide compound solution according to claim 2.

13. The amide compound solution according to claim 6, wherein the cationic surfactant is at least one type selected from among benzethonium chloride, benzalkonium chloride, cetylpyridinium chloride and dequalinium chloride.

14. The amide compound solution according to claim 6, wherein the cationic surfactant is at least one type selected from benzethonium chloride and benzalkonium chloride.

15. The amide compound solution according to claim 6, wherein the amide compound is produced by hydrating a nitrile compound with a biocatalyst.

16. The amide compound solution according to claim 6, wherein the concentration of the amide compound in the amide compound solution is set at 25˜60 mass %.

17. The amide compound solution according to claim 6, wherein the amide compound is acrylamide.
